(19) United States
(12) Patent Application Publication
(10) Pub. No.: US 2014/0349956 A1
(43) Pub. Date: Nov. 27, 2014

(54) MATERIALS AND METHODS FOR DETECTING AND/OR PREDICTING NECROTIZING ENTEROCOLITIS IN INFANTS

(71) Applicant: University of Florida Research Foundation, Incorporated, Gainesville, FL (US)

(72) Inventor: Josef Neu, Gainesville, FL (US)

(21) Appl. No.: 14/372,156

(22) PCT Filed: Jan. 16, 2013
(86) PCT No.: PCT/US2013/021693

§ 371 Date: Jul. 14, 2014

Related U.S. Application Data
(60) Provisional application No. 61/589,015, filed on Jan. 20, 2012.

(51) Int. Cl. G01N 33/68; G01N 33/573

(52) U.S. Cl. CPC G01N 33/6893; G01N 33/6869; G01N 33/573; G01N 2800/065; G01N 2800/38; G01N 2333/908; G01N 2333/8125; G01N 2333/545
USPC 514/40; 435/7.92; 506/9; 435/7.94; 435/7.93; 436/501; 435/7.4

(57) ABSTRACT

The subject invention pertains to materials and methods for diagnosing and/or predicting pathologic infant conditions. A method of the invention comprises obtaining a biological sample from an infant and analyzing the sample to detect at least one protein biomarker of necrotizing enterocolitis (NEC), wherein a patient is diagnosed with NEC or determined to have a likelihood of developing NEC following detection of the biomarker. In another method of the invention, the likelihood of a patient developing NEC is determined. In certain embodiments, treatment is administered to the patient following diagnosis of NEC or determination that the patient has a likelihood of developing NEC.

### CROSS-REFERENCE TO A RELATED APPLICATION

This application claims the benefit of U.S. provisional application Ser. No. 61/589,015, filed Jan. 20, 2012, which is incorporated herein by reference in its entirety.

### BACKGROUND OF THE INVENTION

Necrotizing enterocolitis (NEC) is among the most common and devastating diseases in preterm neonates (Neu, J. & Walker, W. A. Necrotizing enterocolitis. N Engl J Med 2011; 364, 255-64). The mean prevalence of the disorder is about 7% among infants with a birth weight between 500 and 1500 g in the U.S. and Canada based on large, multicenter neonatal databases (Holman R C et al. Necrotising enterocolitis hospitalisations among neonates in the United States. Paediatr Perinat Epidemiol 2006; 20, 498-506; Guillet, R. et al. Association of H2-blocker therapy and higher incidence of necrotizing enterocolitis in very low birth weight infants. Pediatrics 2006; 117, e137-42; Horbar, J D et al. Trends in mortality and morbidity for very low birth weight infants. 1991-1999. Pediatrics 2002; 110, 143-151; and Erasmus H D et al. Enhanced weight gain in preterm infants receiving lactase-treated feeds: a randomized, double-blind, controlled trial. J Pediatr 2002; 141, 532-537).

The estimated rate of death associated with NEC ranges between 20 and 30%, with the highest rate among infants requiring surgery (Fitzgibbons S C et al. Mortality of necrotizing enterocolitis expressed by birth weight categories. J Pediatr Surg 2009; 44, 1072-1075; discussion 1075-1076). Not only is the mortality rate of infants who develop NEC higher, the risk of long-term neurodevelopmental consequences is also significant (Rees, C. M. et al. Neurodevelopmental outcomes of neonates with medically and surgically treated necrotizing enterocolitis. Arch Dis Child Fetal Neonatal Ed 2007; 92, F193-198; and Martin C R et al. Neurodevelopment of extremely preterm infants who had necrotizing enterocolitis with or without late bacteremia. J Pediatr 2010; 157, 751-756 e1).

Early diagnosis of NEC is thus critical to allow timely treatment and intervention. Unfortunately, the pathophysiology of NEC is known to be multifactorial, which renders prediction of risk a major challenge. For example, factors that weigh into the pathophysiology of NEC include intestinal immaturity, bowel stasis, imbalance in microvascular tone, a highly immunoreactive intestinal mucosa, and abnormal intestinal microbial colonization (Young C et al. Biomarkers for infants at risk for necrotizing enterocolitis: clues to prevention? Pediatr Res 2009; 65, 91R-97R; Mai V et al. Fecal microbiota in premature infants prior to necrotizing enterocolitis. PLoS One 2011; 6, e20647; Low J M et al. Proteomic analysis of circulating immune complexes in juvenile idiopathic arthritis reveals disease-associated proteins. Proteomics Clin Appl 2009; 3, 829-840; and Cho, W. C. Contribution of oncoproteomics to cancer biomarker discovery. Mol Cancer 2007; 6, 25).

Because these factors are difficult to monitor, other adjunctive laboratory tests have been utilized to aid in the diagnosis of NEC. Many of these tests require the identification of biomarkers (Young C et al. ibid.). Unfortunately, currently used diagnostic biomarkers such as white blood cell counts, platelet counts, abdominal x-rays, and blood cultures are costly, invasive and/or non-specific.

In one attempt to develop a more specific test for NEC, intestinal microbiota was evaluated to identify at-risk infants by using early stool samples (including meconium) of preterm infants (Mai V et al. ibid.). It was demonstrated that changes in the stool microbiota in these infants occur prior to the onset of NEC. However, defecation by preterm infants is often sporadic and unpredictable making microbiota based early detection difficult to implement in a clinical setting.

Among the previous attempts to predict NEC, some investigators proposed the use of scoring systems that can predict NEC likelihood (Alfaleh, K. & Bassler, D. Probiotics for prevention of necrotizing enterocolitis in preterm infants. Cochrane Database Syst Rev. 2008; 23, CD005496). In their study, a total of 944 patients participating in the Trial of Indomethacin Prophylaxis in Preterms (TIPP) trial (Schmidt B, Davis P, Moddemann D, Ohlsson A, Roberts R S, Saigal S et al. Long-term effects of indomethacin prophylaxis in extremely-low-birth-weight infants. N Engl J Med 2001; 344, 1966-1972) had their information regarding their hospital course collected prospectively, including culture-proven sepsis, meningitis, stage II or III NEC, bronchopulmonary dysplasia (BPD), brain injury, and severe retinopathy. Of 414 infants, 44% had at least one episode of infection or NEC, but there was no epidemiologic information that could predict NEC. For this reason, it has been suggested that the development of non-invasive biomarkers to help predict and prevent disease in neonates represents a significant need (Maresso, K. & Broeckel, U. The role of genomics in the neonatal ICU. Clin Perinatol. 2009; 36, 189-204).

Recent studies have utilized genomics and proteomics. Using a proteomic approach, one group of investigators suggested the novel ApoSAA score to help differentiate NEC and sepsis in 77 infants compared to 77 control infants (Ng P C, Ang I L, Chiu R W, Li K, Lam H S, Wong R P et al. Host-response biomarkers for diagnosis of late-onset septicemia and necrotizing enterocolitis in preterm infants. J Clin Invest. 2010; 20:2989-3000). While the study was able to establish the novel ApoSAA score to help diagnose NEC and sepsis, it was limited to diagnosis at the time of presentation and did not distinguish NEC from sepsis. Although these diseases can occur simultaneously, this method would not allow for early detection or differentiation allowing for potential preventive measures to be instituted. This method also required the invasive procedure of obtaining blood for plasma analysis, which can be painful and has potential associated complications.

In another study searching for a NEC biomarker (Thuijls G, Derikx J P, van Wijck K, Zimmermann L J, Degraeuwe P L, Mulder T L et al. Non-invasive markers for early diagnosis and determination of the severity of necrotizing enterocolitis. Ann Surg. 2010; 251:1174-1180) a non-invasive approach used collection of daily urine samples from infants thought to be at risk for the development of NEC. Urinary intestinal fatty acid binding protein (I-FABP), claudin-3, and calprotectin were identified as potential biomarkers to aid in the diagnosis of NEC. While the study was able to identify these proteins as potential diagnostic markers, it failed to show that I-FABP, claudin-3, or calprotectin could be used as predictive biomarkers before the onset of NEC.

Thus, better biomarkers are needed for bedside diagnostic/prediction of NEC, sepsis or other pathological conditions in infant patients.

### BRIEF SUMMARY OF THE INVENTION

The subject invention provides novel assays based on the identification of biomarkers that are predictive and/or diagnostic of diseases in infants. Specifically, the subject invention provides minimally invasive methods for identifying biomarkers that are predictive and/or diagnostic of NEC and/or sepsis. In one embodiment, the subject invention provides bedside assays for use in patients admitted to the neonatal intensive care unit.

Using a proteomic platform, several proteins were identified that are altered (increased or decreased) prior to the onset of NEC in susceptible neonates. Thorough analysis of these proteins revealed their molecular functions and biologic functions. The pathways in which these proteins are involved were also mapped.

These proteins have been identified to be specific biomarkers for determining whether a patient has or is predisposed to a disease, particularly NEC. In one embodiment, the methods of the invention employ assays for the detection of protein biomarkers of NEC including, but not limited to, IL-1RA, peroxiredoxin-1 and alpha 1 anti-trypsin. Preferably, the protein biomarker of NEC is IL-1RA. Sensitive and specific assays for protein biomarkers of NEC include, but are not limited to, immunoassays, such as enzyme immunoassays and radioimmunoassays.

In accordance with the subject invention, biological samples are obtained from infants. Preferably, the biological samples are obtained from preterm or neonatal infants. Typical biological samples include, but are not limited to, serum, urine, and mucosa. Samples from the mouth represent an attractive method to obtain patient samples non-invasively. In one embodiment, the biological sample is obtained from within the mouth. Preferably, the samples are cheek (buccal) swab samples.

In a preferred embodiment, the method of the invention comprises the steps of: obtaining a buccal biological sample from a neonatal patient; analyzing the sample to detect at least one protein biomarker of NEC; and, based on the analysis, determining whether the patient has, or is predisposed to have, NEC. More preferably, the level of protein(s) is detected to determine whether the patient has or is predisposed to NEC. The protein is preferably selected from the group consisting of: peroxiredoxin 1, IL-1RA, and alpha 1 anti-trypsin.

In certain embodiments, following diagnosis of NEC in a patient, further steps are taken to treat the patient.

In a further embodiment, a confirmation step is performed to confirm whether the patient has, or is predisposed to have, NEC. The confirmation step includes conducting at least one further diagnostic test for NEC. Current diagnostic tests for NEC include, but are not limited to, white blood cell counts, platelet counts, abdominal x-rays, blood cultures, analysis of intestinal microbiota, and analysis of urinary intestinal fatty acid binding protein (I-FABP), claudin-3, and calprotectin.

There are currently several potential interventions for NEC in infant patients. The methods of the subject invention facilitate identification of patients to whom these preventive and treatment strategies should be utilized. In one embodiment, following determination of a likelihood of developing NEC in a patient, steps are taken to treat, deter and/or prevent NEC development.

### BRIEF DESCRIPTION OF THE DRAWINGS

The file of this patent contains at least one drawing executed in color. Copies of this patent with color drawings(s) will be provided by the Patent and Trademark Office upon request and payment of the necessary fee.

FIG. 1. Differential protein expression of baby buccal swab as revealed by 2-D DIGE analysis. Protein extract from NEC and normal baby buccal swabs were labeled with Cy3 and Cy5, respectively. A mixed internal reference combining all the proteins from both NEC and normal and their replicates was labeled with an additional Cy2 dye, and was included in all gels. Non linear IPG strips (3 to 11) were used for IEF and 8 to 16% Tris-glycine SDSPAGE for the second dimension. Both normal (FIG. 1A) and NEC (FIG. 1B) images show the spot map on which circled spots marked with spot number indicate those proteins identified with protein abundance changes when comparing NEC to normal with 95% confidence level. Numbers in the figure correlate with those included in Table 1 described herein.

FIG. 2: Western blot for peroxiredoxin 1 (A), IL-IRA (B) and α-1 antitrypsin (C). Equal amounts of protein samples (15 μg) were loaded and separated on 4-20% SDS-PAGE gels and transferred to PVDF membranes. The images represent the protein expression after probed using different primary antibodies, rabbit anti-peroxidorexin 1 (FIG. 2A), rabbit anti-IL-1RA antagonist (FIG. 2B), and mouse anti-alpha 1 antitrypsin (FIG. 2C), respectively. Protein band density was analyzed using ImageQuant software. Bar graphs show relative protein concentrations that were quantitated and normalized by total protein loading with SYPRO Ruby staining.

FIG. 3: Molecular function, Biological Process and Cellular localization of Altered NEC Protein Data Set. Gene Ontology analysis of the NEC altered protein data set was performed to further characterize these proteins. Thirty-eight assignments were obtained for molecular function_annotations which were sorted into 6 categories; proteins were mainly distributed among binding activity (˜40%), enzyme regulatory functions (21%), and catalytic activity (36%), and other were involved in structural and anti-oxidant activity (FIG. 3A).

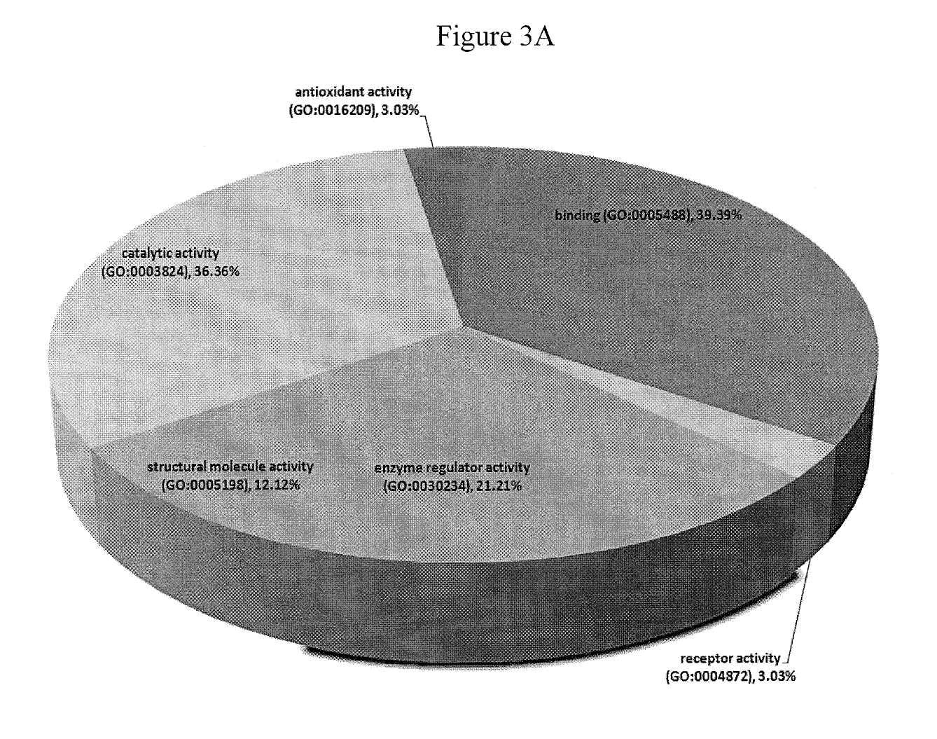

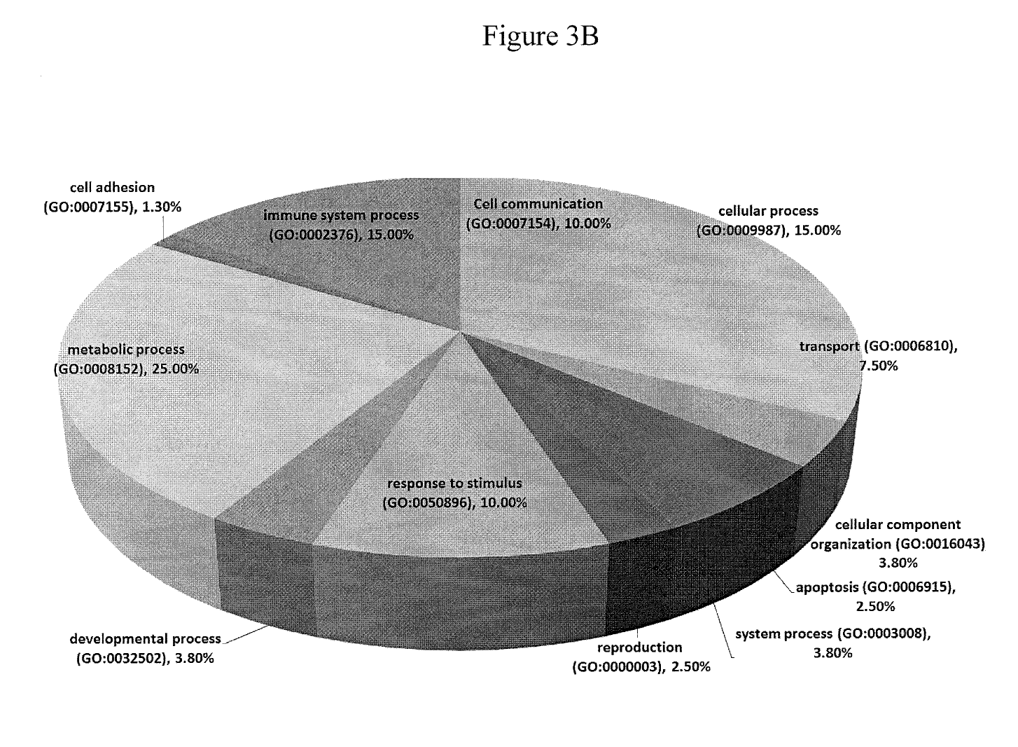

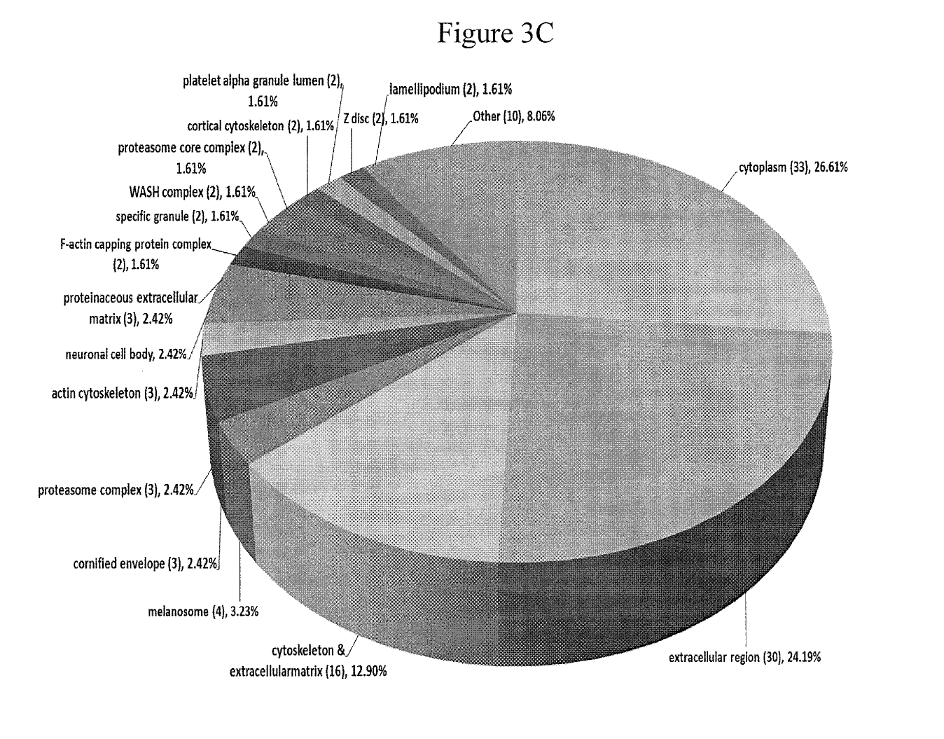

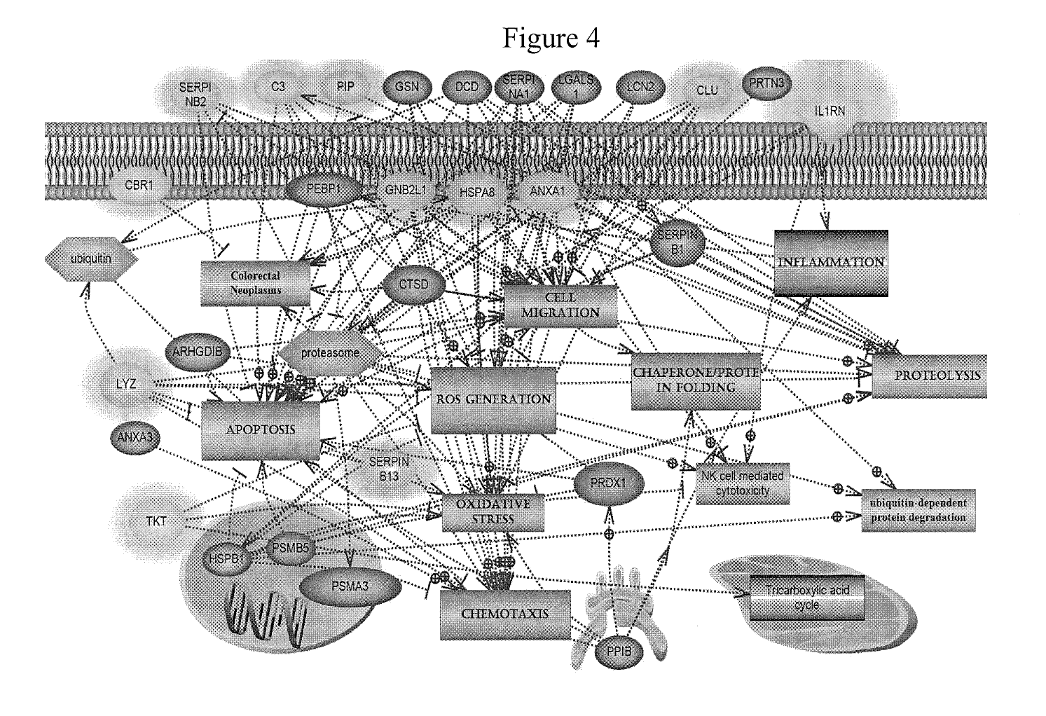

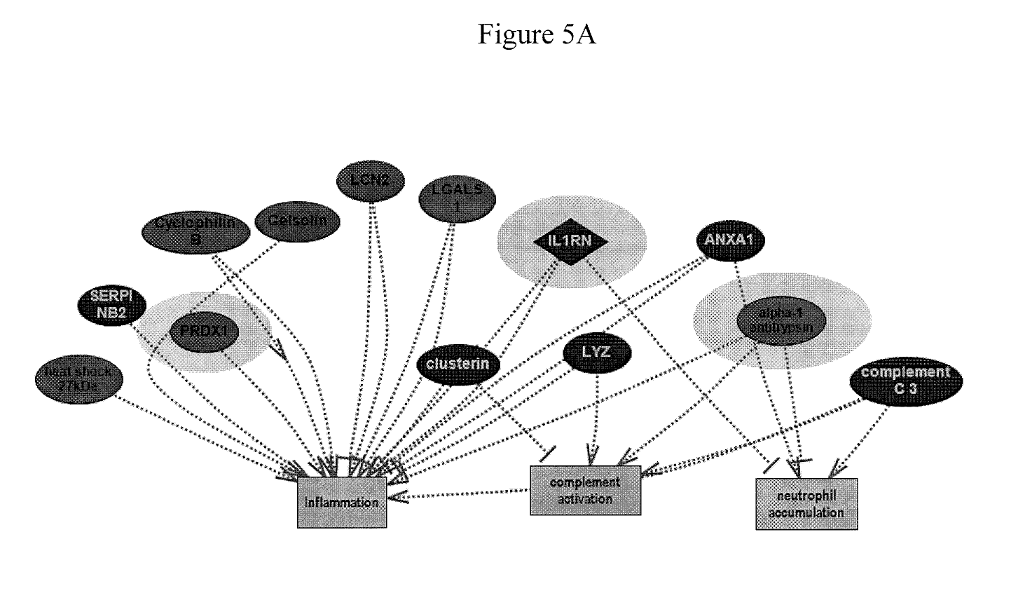

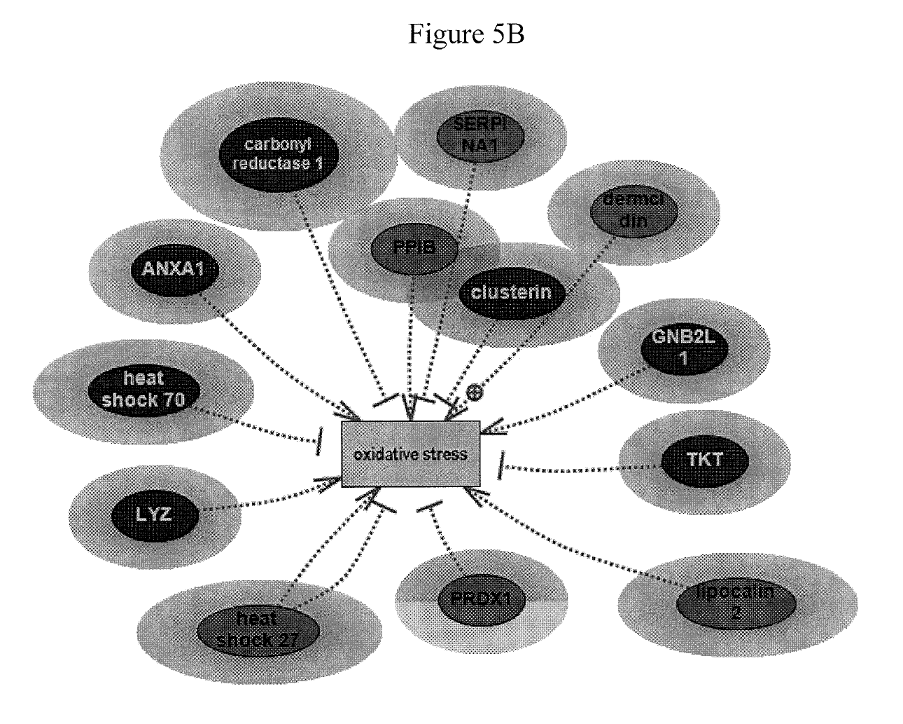

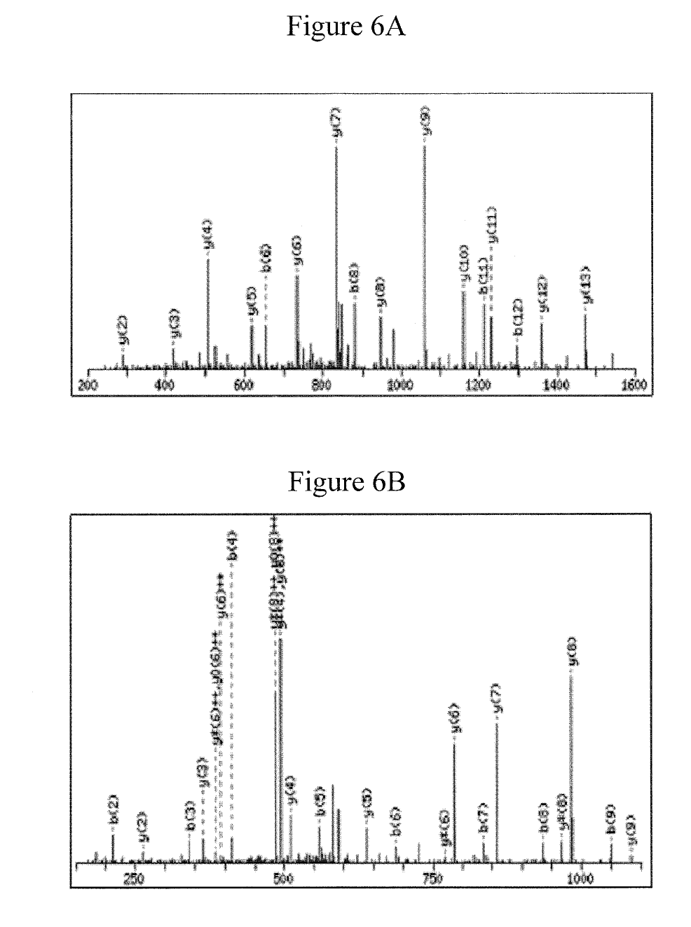

For the biological process annotations, proteins were grouped into 12 broad categories of biological process classifications. Whereas most of the altered proteins (25%) are involved in metabolic process, 25% of all proteins exhibited immune response along with stimulus response activity; cellular process and communication represented 22% of the total biological process (FIG. 3B).

Cellular localizations of the altered proteins identified were categorized into 18 different cellular compartments. Approximately, 50% of the proteins were localized in the soluble cellular fraction—cytoplasm and extracellular region (26.6% and 2%, respectively), compared to 13% of cytoskeletal and other organelle specific localizations. This may be due to the capabilities of 2-D gel separation platform which is more biased towards soluble proteins (FIG. 3C).

FIG. 4: Global Analysis of altered Pathways and Networks in the NEC samples. Using Pathway Studio analysis, several pathways were identified based on the altered proteome; these pathways are believed to be central to the pathogenesis of NEC. These processes include inflammation, cell death, oxidative stress, cell migration and apoptosis. The red color shows up-regulated proteins prior to NEC onset. The network was generated using the “direct interaction” algorithm with the filters of “cellular process; entity and relation type as regulation” to map altered pathways regulated by the identified (increased and decreased) proteins. A more in-depth analysis of two of these pathways is discussed in supplemental data.

FIG. 5: Sub-Networks Enriched Analysis of the NEC Altered Proteins. In this analysis, enriched sub-networks of altered proteins using the identifiers “Cellular Process/Regulation” filters and downstream directionality identified individual pathway-protein components. A sub-network enriched analysis showed that there are 13 proteins involved in the inflammatory process including heat shock 27, alpha1-antitrypsin, lectin, serpin peptidase inhibitor, complement C3, peroxiredoxin 1, clusterin, guanine nucleotide binding protein, lysozyme, gelsolin, annexin A1, lipocalin 2 and IL1 RN. These proteins were mapped to inflammation, complement activation and neutrophil activation (FIG. 5A). Similarly, oxidative stress response was among the altered pathways implicated in NEC. 13 proteins were shown to be regulating the dynamics of oxidative stress response either by activation and/or inhibition. These included heat shock 27, transketolase, alpha1-antitrypsin, peroxiredoxin 1, clsuterin, annexin A1, lipocalin 2, carbonyl reductase 1, heat shock 70, alpha1-antitrypsin, lysozyme, guanine nucleotide binding protein, cyclophillin B. (FIG. 5B). Highlighted proteins indicate differential expression validation by Western blot.

FIG. 6: Examples of the mass spectra for some of the proteins of interest. (FIG. 6A) MS/MS ion series showing a peptide sequence LQLEAVNITDLSENR, which by itself identifies the protein in interleukin-1 receptor antagonist: (FIG. 6B) MS/MS ion series showing a peptide sequence LVQAFQFTDK, which by itself indentified the protein peroxiredoxin. Details provided in Table 1.

### DETAILED DESCRIPTION OF THE INVENTION

The subject invention provides non-invasive and sensitive methods for the early diagnosis, prognosis, and/or monitoring of pathologic conditions by detecting specific biomarkers in a sample. In one embodiment, the method of the invention comprises the steps of: obtaining a biological sample from an infant; analyzing the sample to detect at least one protein that is a biomarker of the pathologic condition; and determining, based on the analysis of the sample, whether the infant has or is predisposed to the pathologic condition.

The methods of the invention include obtaining a biological sample from an infant and detecting a biomarker of NEC in the sample. In preferred embodiments, the infant is a neonate, which encompasses all infants in at least the first 28 days of birth including, for example, premature infants, postmature infants, and full term infants. More preferably, the infant is a neonate.

A biological sample can be serum, urine or mucosal. A particularly advantageous biological fluid for performing the non-invasive diagnostic methods of the present invention is a biological sample from the mouth.

In one embodiment, a non-invasive method of swabbing the buccal epithelium is provided to collect saliva for diagnosis or prediction of a pathological condition, such as NEC. Saliva, a biologic material that is easy to obtain non-invasively and in a repeatable fashion, has previously been described as “the mirror of the body” and the “perfect medium to be explored for health and disease surveillance” (Segal A, Wong D T. Salivary diagnostics: enhancing disease detection and making medicine better. Eur J Dent Educ 2008; 12 Suppl 1, 22-29).

Obtaining biologic material by swabbing the buccal epithelium in infants, particularly neonates, provides a novel way to investigate the complex physiology likely occurring prior to the onset of NEC as well as provide a means for minimally invasive diagnosis of NEC. Being relatively non-invasive, this collection method allows investigators to evaluate not only whether the infant has or is predisposed to NEC but also to evaluate what is happening in the plasma (as saliva is a by-product of blood) and in the intestine (since the buccal epithelium and intestinal epithelium are from the similar embryologic origins).

The biological sample from the infant patient may be assayed for at least one protein biomarker of NEC using any appropriate analytical method for proteins. Sensitive and specific assays include, for example, immunoassays that are described in more detail herein. In certain embodiments, the level of the protein(s) in the sample is determined using common assays (such as with immunoassays and protein arrays). Information regarding the presence and/or level of protein associated with NEC is then used, in accordance with the invention, to determine whether a patient has, or is predisposed to having, NEC.

Using a proteomic approach, several biomarkers were identified and characterized from buccal swabs of very low birthweight neonates. 2-DIGE results were utilized to map global interactions that identified a number of NEC-relevant pathways (inflammation, colorectal cancer, oxidative stress, and chemotaxis etc). Two major pathways (inflammation, and oxidative stress; FIG. 5A/FIG. 5B) that are known to be involved in NEC pathogenesis were identified. A sub-network enriched analysis showed that there are 13 proteins involved in the inflammatory process (e.g., alpha1-antitrypsin, complement C3, peroxiredoxin 1, heat shock 27, cyclophillin B and IL1 RA see FIG. 5A) which were mapped to inflammation, complement activation and neutrophil accumulation. Similarly, oxidative stress response has also been implicated to be major in NEC and is reflected in FIG. 5B. Also, 13 proteins were shown to be regulating the dynamics of oxidative stress response either by activation and/or inhibition which has a major implication on cell death process.

According to the subject invention, biomarkers of NEC include, but are not limited to, peroxiredoxin 1, IL-1RA, and alpha 1 anti-trypsin. More preferably, a biomarker of NEC that is detected using the methods of the invention is IL-1RA.

In the methods of the present invention, the diagnosis as well as determination of the likelihood of having NEC is important for clinical intervention/treatment. This “negative” diagnosis, such as determination that the patient does not have NEC or that the patient is not predisposed to having NEC, is of great significance since it eliminates the need of subjecting a patient to unnecessary treatment or intervention, which could have potential side-effects, or may otherwise put the patient at risk.

In further embodiments, diagnosis of a particular pathological condition, such as NEC and/or sepsis, can be based on characteristic differences (unique expression signatures) between a normal proteomic profile of a biological sample from a normal infant, and a proteomic profile of the same biological fluid obtained under the same circumstances, where the disease or pathologic condition (such as NEC and/or sepsis) is diagnosed as present in the infant. The unique expression signature can be any unique feature or motif within the proteomic profile of a test or reference biological sample that differs from the proteomic profile of a corresponding normal biological sample obtained from the same type of source, in a statistically significant manner. For example, if the proteomic profile is presented in the form of a mass spectrum, the unique expression signature is typically a peak or a combination of peaks that differ, qualitatively or quantitatively, from the mass spectrum of a corresponding normal sample. Thus, the appearance of a new peak or a combination of new peaks in the mass spectrum, or any statistically significant change in the amplitude or shape of an existing peak or combination of existing peaks, or the disappearance of an existing peak, in the mass spectrum can be considered a unique expression signature. When the proteomic profile of the test sample obtained from an infant patient is compared with the proteomic profile of a reference sample comprising a unique expression signature characteristic of a disease/condition such as NEC or sepsis, the patient is diagnosed with such disease/condition if it shares the unique expression signature with the reference sample.

Alternatively or in addition, the proteomic profile of the test sample may be compared with the proteomic profile of a reference sample obtained from a biological fluid of an infant patient independently diagnosed with NEC or sepsis. In this case, the patient is diagnosed with NEC or sepsis if the proteomic profile of the test sample shares at least one feature, or a combination of features representing a unique expression signature, with the proteomic profile of the reference sample.

In a further embodiment, a confirmation step is performed to confirm whether the patient has, or is predisposed to have, NEC. The confirmation step includes conducting at least one further diagnostic test for NEC. Current diagnostic tests for NEC include, but are not limited to, white blood cell counts, platelet counts, abdominal x-rays, blood cultures, analysis of intestinal microbiota, and analysis of urinary intestinal fatty acid binding protein (I-FABP), claudin-3, and calprotectin.

Proteomic techniques were used to identify biomarkers of pathologic conditions in infants, preferably NEC and/or sepsis. For example, samples can be analyzed using advanced multi-dimensional proteomic separation techniques coupled with mass spectrometry analysis. Similar techniques have been previously utilized in identifying disease biomarkers in other medical areas including: immunology (Low J M et al. ibid.), cancer (Cho, W. C. ibid.), obstetrics (Ho J, et al. Novel breast cancer metastasis-associated proteins. J Proteome Res. 2009; 8, 583-594), and psychiatry (Kobeissy F H et al. Psychoproteomic analysis of rat cortex following acute methamphetamine exposure. J Proteome Res 2008; 7, 1971-1983). Those skilled in the art will recognize many methods and materials similar or equivalent to those described herein, which could be used in the practice of the present invention.

According to the subject invention, a representation of the expression pattern of at least one or a plurality of proteins in a biological sample, e.g. a biological fluid obtained from a cheek swab at a given time, is obtained. This representation, also known as a proteomic profile, can, for example, be represented as a mass spectrum, but other representations based on any physicochemical or biochemical properties of the proteins are also included. The proteomic profile may, for example, be based on differences in the electrophoretic properties of proteins, as determined by two-dimensional gel electrophoresis, e.g. by 2-D PAGE, and can be represented, e.g., as a plurality of spots in a two-dimensional electrophoresis gel. Differential expression profiles may have important diagnostic value, even in the absence of specifically identified proteins. Single protein spots can then be detected, for example, by immunoblotting, multiple spots or proteins using protein microarrays. The proteomic profile typically represents or contains information that could range from a few peaks to a complex profile representing 50 or more peaks.

Typically, protein patterns (proteomic profile) of samples from different sources, such as normal biological fluid (normal sample) and a test biological fluid (test sample), are compared to detect proteins that are up- or down-regulated in a disease. These proteins can then be excised for identification and full characterization, e.g., using peptide-mass fingerprinting and/or mass spectrometry and sequencing methods, or the normal and/or disease-specific proteome map can be used directly for the diagnosis of the disease of interest (such as NEC or sepsis), or to confirm the presence or absence of the disease.

According to one embodiment of the invention, following diagnosis of NEC in a patient based on detection of at least one protein biomarker of NEC in a patient's biological sample, the patient is treated for NEC. Treatment for NEC can include operative and/or nonoperative treatment. Operative treatments include, but are not limited to, resecting perforated and/or necrotic intestine, enterostomy closure, and peritoneal drainage. Nonoperative treatments include, but are not limited to, stopping enteral feedings, performing nasogastric decompression, intravenous fluids, total parenteral nutrition, and initiating broad-spectrum antibiotics. Antibiotics can be any one or a combination of ampicillin, gentamicin, and clindamycin (or metronidazole).

In another embodiment of the invention, following determination that a patient has a likelihood of developing NEC, the patient is treated. Such prophylactic treatment can include supplementing the patient's diet with any one or combination of the following: enteral immunoglobulin A, glucocorticoids, oral antibiotics, and/or PAF antagonists.

In one embodiment, analysis of buccal swab samples from infants is conducted using a 2-dimensional difference gel electrophoresis (2D-DIGE) LC-MS/MS proteomics platform to identify predictive biomarkers of NEC. According to the subject invention, biomarkers of NEC include, but are not limited to, peroxiredoxin 1, IL-1RA, and alpha 1 anti-trypsin. More preferably, a biomarker of NEC that is detected using the methods of the invention is IL-1RA.

### Immunoassays

The diagnostic assay of the present invention can also be performed in the form of various immunoassay formats, which are well known in the art. There are two main types of immunoassays, homogenous and heterogenous. In homogenous immunoassays, both the immunological reaction between an antigen and an antibody and the detection are carried out in a homogenous reaction. Heterogenous immunoassays include at least one separation step, which allows the differentiation of reaction products from unreacted reagents.

ELISA is a heterogenous enzyme-linked immunoabsorbent assay, which has been widely used in laboratory practice since the early 1970's. The assay can be used to detect antigens in various formats.

In the “sandwich” format the antigen being assayed is held between two different antibodies. In this method, a solid surface is first coated with a solid phase antibody. The test sample, containing the antigen (i.e. a diagnostic protein) being measured, is then added and allowed to react with the bound antibody. Any unbound antigen is washed away. A known amount of enzyme-labelled antibody is then allowed to react with the bound antigen. Any excess unbound enzyme-linked antibody is washed away after the reaction. The substrate for the enzyme used in the assay is then added and the reaction between the substrate and the enzyme produces a colour change. The amount of visual colour change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labelled antigen and both compete for binding to an anti-antigen antibody attached to a solid surface. Excess free enzyme-labelled antigen is washed off before the substrate for the enzyme is added. The amount of color intensity resulting from the enzyme-substrate interaction is a measure of the amount of antigen in the sample tested.

In certain related immunoassays, the antigens are radiolabelled (also referred to as a radioimmunoassay).

Homogenous immunoassays include, for example, the Enzyme Multiplied Immunoassay Technique (EMIT), which typically includes a biological sample comprising the compound or compounds to be measured, enzyme-labeled molecules of the compound(s) to be measured, specific antibody or antibodies binding the compound(s) to be measured, and a specific enzyme chromogenic substrate. In a typical EMIT excess of specific antibodies is added to a biological sample. If the biological sample contains the proteins to be detected, such proteins bind to the antibodies. A measured amount of the corresponding enzyme-labelled proteins is then added to the mixture. Antibody binding sites not occupied by molecules of the protein in the sample are occupied with molecules of the added enzyme-labelled protein. As a result, enzyme activity is reduced because only free enzyme-labelled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the Ag-enzyme complex is Ab-bound makes the EMIT a unique system, enabling the test to be performed without a separation of bound from unbound compounds as is necessary with other immunoassay methods.

Part of this invention is also an immunoassay kit comprising, in separate containers, (a) monoclonal or polyclonal antibodies having binding specificity for the polypeptides used in the diagnosis of a disease/condition, such as NEC; and (b) anti-antibody immunoglobulins. This immunoassay kit may be utilized for the practice of the various methods provided herein. The monoclonal or polyclonal antibodies and the anti-antibody immunoglobulins may be provided in an amount of about 0.001 mg to 100 grams, and more preferably about 0.01 mg to 1 gram. The anti-antibody immunoglobulin may be a polyclonal immunoglobulin, which may be labeled prior to use by methods known in the art. The monoclonal or polyclonal antibodies specific for the polypeptides used in the diagnosis of a particular disease/condition, such as NEC, can be adapted to rapid spot quantification utilizing calorimetric or charge state detection using suitable reading devices available in the field.

### Protein Arrays

In recent years, protein arrays have gained wide recognition as a powerful means to detect proteins, monitor their expression levels, and investigate protein interactions and functions. They enable high-throughput protein analysis, when large numbers of determinations can be performed simultaneously, using automated means. In the microarray or chip format, that was originally developed for DNA arrays, such determinations can be carried out with minimum use of materials while generating large amounts of data.

Although proteome analysis by 2D gel electrophoresis and mass spectrometry is very effective, it does not always provide the needed high sensitivity and thus might miss many proteins that are expressed at low abundance. Protein microarrays, in addition to their high efficiency, provide improved sensitivity.

Protein arrays are formed by immobilizing proteins on a solid surface, such as glass, silicon, micro-wells, nitrocellulose, PVDF membranes, and microbeads, using a variety of covalent and non-covalent attachment chemistries well known in the art. The solid support should be chemically stable before and after the coupling procedure, allow good spot morphology, display minimal nonspecific binding, should not contribute a background in detection systems, and should be compatible with different detection systems.

In general, protein microarrays use the same detection methods commonly used for the reading of DNA arrays. Similarly, the same instrumentation as used for reading DNA microarrays is applicable to protein arrays.

Thus, capture arrays (e.g. antibody arrays) can be probed with fluorescently labelled proteins from two different sources, such as normal and diseased biological fluids. In this case, the readout is based on the change in the fluorescent signal as a reflection of changes in the expression level of a target protein. Alternative readouts include, without limitation, fluorescence resonance energy transfer, surface plasmon resonance, rolling circle DNA amplification, mass spectrometry, resonance light scattering, and atomic force microscopy.

For further details, see, for example, Zhou H, et al., Trends Biotechnol. 19:S34-9 (2001); Zhu et al., Current Opin. Chem. Biol. 5:40-45-(2001); Wilson and Nock, Angew Chem Int Ed Engl 42:494-500 (2003); and Schweitzer and Kingsmore, Curr Opin Biotechnol 13:14-9 (2002). Biomolecule arrays are also disclosed in U.S. Pat. No. 6,406,921, issued Jun. 18, 2002, the entire disclosure of which is hereby expressly incorporated by reference.

### Mass Spectrometry Based Assays

Recent advances in mass spectrometry (Anderson L. and Hunter C. L., Mol Cell Proteomics, 2006 April; 5(4):573-88) enable quantification of specific proteins and polypeptides by monitoring the specific ions by mass selection. These assays use mass selection to provide absolute specificity, first selection (MS1) involves capture of parent ion and, second step captures specific fragment of the parent ion (Multiple reaction monitoring, MRM) detection and quantification. With appropriate standards for a specific protein, MRM assays could provide a reliable quantification of analytes to monitor various disease specific biomakers. Monoclonal or polyclonal antibodies for markers of maternal fetal diseases can be used to capture and analyze by MRM assays.

Following are examples which illustrate procedures for practicing the invention. These examples should not be construed as limiting. All percentages are by weight and all solvent mixture proportions are by volume unless otherwise noted.

### Methods:

### Study Design and Patient Identification

This study was approved by the Investigational Review Board of the University of Florida. Preterm infants from three University of Florida affiliated hospitals were screened for study enrollment. Patients who met study criteria and had informed consents signed by the parents were included in this study. The inclusion criteria included neonates with birth weights of less than or equal to 1,250 grams and with gestational ages less than or equal to 32 weeks. Infants having known congenital malformations of the intestine such as gastroschisis or atresias, and infants with lethal conditions were excluded. After enrollment, buccal samples were collected at weekly intervals (within the 7 days of each week of life, but no sooner than 3 days after the prior sample). Patients were considered to have met the criteria for diagnosis of NEC (Modified Bell Stage 2 or 3 with radiologic pneumatosis intestinalis and/or portal venous gas; or direct intraoperative confirmation of intestinal pathology) (Bell M J, Ternberg J L, Feigin R D, Keating J P, Marshall R, Barton L. et al. Neonatal necrotizing enterocolitis. Therapeutic decisions based upon clinical staging. Ann Surg 1978; 187, 1-7; Walsh M C, Kilegman R M. Necrotizing Enterocolitis: treatment based on staging criteria. Pediatr Clin North Am 1986; 33, 179-201). Control infants were selected and matched to NEC case infants using gestational age, birth weight, birth center (to avoid confounders of practice variation), date of birth (+/−2 months to avoid variable infectious agent seasonality), and predominant enteral nutrient (breast milk vs. formula).

### ABBREVIATIONS

Necrotizing Enterocolitis (NEC), Neonatal Intensive Care Units (NICUs), International Protein Index (IPI), Heparin-Binding Epidermal Growth Factor-like growth factor (HB-EGF), Bronchopulmonary Dysplasia (BPD), 2-dimensional gel electrophoresis (2D-DIGE), Intestinal Fatty-Acid Binding Protein (I-FABP).

### Sample Choice

Samples used were those from NEC cases that were collected in the weeks prior to the onset of NEC (3 weekly samples from each patient) and from matched time points in controls. The 3 weekly samples were then pooled to increase the amount of protein contained for each patient.

### Buccal Swab Collection

A Cytobrush Plus Cell Collector (CooperSurgical Inc., Trumbull, Conn.) was placed in the mouth and was twisted 360 degrees on both cheeks and also on the tongue to maximize protein collected. The brush was then washed in a 2 ml cryovial containing 1 ml phosphate buffered saline. The samples were then immediately placed in a −80 C freezer for later analysis.

### Buccal Swab Protein Extraction

While still frozen, 10×RIPA buffer (10% TRITON X-100, 5% sodium deoxycholate, 1% SDS, 10 mM EDTA) with protease inhibitor (Calbiochem 539134) was added to buccal swab and was agitated at cold for 1 hour. After sonication for three times each for 10 seconds and after clarification at 20,000 g for 20 minutes at cold, supernatants from multiple samples from each patient were combined (total of 6 resulting samples, one pooled sample from each patient) and precipitated with 9 volumes of ice cold 10% TCA/Acetone overnight at −20 C. The resulting protein pellet was recovered by centrifugation at 20,000 g for 20 min at cold and was washed twice with 80% ethanol then twice with 80% acetone. The protein pellet was then air dried at cold for 5 minutes and dissolved in DIGE labeling buffer (8M urea 2M thiourea, 4% CHAPS, 20 mM Tris (pH8.5). Benzonase (Novagen) was added to each dissolved extract to digest large molecules of nucleic acid. The resulting solution was then clarified at 40,000 g for 30 minutes at 15° C. before protein quantification assay. Protein concentration of these samples was determined with EZQ Protein Quantification Kit (Invitrogen) and ova albumin as standard.

### Protein-CyDye Labeling

Protein labeling with CYDYE was performed using the CYDYEe technology developed by GE Healthcare. After adjusting sample solution to pH 8.5, each protein sample was covalently linked to a different CYDYE fluorophore, such as Cy2 to reference (a mixture of equal amounts of protein extracts from all samples presenting in this project), Cy3 to control, and Cy5 to NEC. In each case 100 ug of protein was labeled with 400 pmol CYDYE for 30 min in dark in cold, then quenched with 1 ul of 10 mM lysine.

### 2D-DIGE Experimental Design

To ensure statistical significance for data obtained from control and NEC cases, the following experimental design was applied. Equal amounts (100 μg) of Cy3-control, Cy5-NEC, and Cy2-reference mixture were loaded per gel, resulting in 300 ug total protein loaded in each gel. For biological variation evaluation, three separate gels each including a different set of samples were performed to evaluate the protein abundance variation statistically. To minimize dye difference if there was any, Cy3/Cy5 dye swapping between control and NEC was performed also.

### 2-D Gel Electrophoresis

Three different CYDYE labeled samples were mixed and brought to 500 ul volume with IEF buffer (8 M urea 2M thiourea 4% CHAPS 100 mM DDT 0.5% IPG buffer pH 3 to 11) before passively rehydrating a IPG pH 3 to 11 non linear strip (GE Healthcare). 3 strips were used to separate proteins at 19° C. on an IPGphor Unit (GE Healthcare) with voltage ramping up to and held at 10000 Volt for a total 60 kVh.

After completion of IEF, the strips were first equilibrated with reducing buffer (50 mM Tris-HCl (pH 6.8), 6 M Urea, 30% (v/v) glycerol, 2% (w/v) SDS 100 mM DTT), then were equilibrated in alkylation buffer (50 mM Tris-HCl (pH 6.8), 6 M Urea 30% (v/v), glycerol 2%, SDS 2.5% idoacetamide), both equilibrations were held at room temperature in dark for 15 minutes. After equilibration, the strip was transferred and mounted on top a 24×24 cm 8 to 16% Tris Glycine polyacrylamide gel (Jule) under a layer of warm 0.5% agarose made in SDS electrophoresis running buffer. Electrophoresis was carried out in Ettan Daltsix Unit (GE Healthcare) at 12 C at 10 mA/gel for one hour then overnight at constant current at 12 mA/gel and a limit of 150 V until the dye front reached the bottom of the plate.

### Image Acquisition and Data Analysis

Immediately after gel electrophoresis, CYDYE labeled proteins in gel were scanned using a Typhoon 9400 Variable Mode Imager (GE Healthcare). The excitation/emission wavelengths for Cy2, Cy3 and Cy5 were 488/520, 532/580 and 633/670 nm respectively. Three images per gel (internal standard, control and experimental) were acquired.

The digital image information acquired was then analyzed with DeCyder 2D software, version 7.0, (GE Healthcare). All spots present in all images in each individual gel were co-detected, matched, and normalized with the DIA (Differential In-Gel Analysis) Module within the software. Information from replicate gels was analyzed with the BVA (Biological Variation Analysis) Module. In the BVA, a master gel was created with information from all gels, including matching multiple images from different gels to provide statistical data on differential protein expression levels between control and experimental groups. There were 2000 spots detected and matched. Interesting spots were selected by setting the fold difference threshold to 1.5 fold (any protein spots from the experimental group that was expressed above or below 1.5 fold when compared with spots from the control group were selected). These spots are listed in Table 1. A pick list was made after filtering the spot information based on matching quality, appearance in all gels (6 out of 9 images in this case), and statistical confidence when Student's t-test p value was less than 0.05.

TABLE 1
Change and identification of proteins compared between normal and NEC babies

| spot | | Fold | | mass | mass | | | | unique |
|---|---|---|---|---|---|---|---|---|---|
| # | Protein Name | case/normal | p-value | kDa/pI | kDa/pI | IPI number | score | coverage | peptides |
| 3013 | Interleukin-1 receptor | −3.15 | 0.029 | 16.23/5.01 | 20.04/5.82 | IPI00174541 | 256 | 28 | 5 |
| | antagonist (IL-1RA) | | | | | | | | |
| 2971 | | −2.19 | 0.01 | 18.09/5.05 | | | 525 | 24 | 4 |
| 2892 | Peroxiredoxin-1 | 4.18 | 0.004 | 23.53/8.59 | 22.09/8.27 | IPI00000874 | 274 | 39 | 8 |
| 998 | Isoform 1 of Alpha-1- | 3.18 | 0.045 | 72.77/5.53 | 46.72/5.37 | IPI00553177 | 117 | 6 | 3 |
| | antitrypsin | | | | | | | | |
| 1005 | | 3.48 | 0.021 | 72.49/5.59 | | | 127 | 6 | 4 |
| 1203 | Clusterin isoform | −2.16 | 0.0087 | 65.50/7.62 | 52.48/5.88 | IPI00291262 | 140 | 8.91 | 3 |
| | (apolipoprotein J) | | | | | | | | |
| 1146 | | −1.95 | 0.019 | 67.23/7.24 | | | 159 | 8.9 | 3 |
| 2777 | Proteosome subunit alpha type 2 | 1.18 | 0.047 | 26.04/5.05 | 25.90/6.91 | IPI00219622 | 85 | 13.2 | 2 |
| 854 | Gelsolin (isoform 2-cytoplasmic) | 1.78 | 0.019 | 81.26/5.61 | 80.62/5.58 | IPI00646773 | 698 | 22 | 11 |
| 855 | | 1.76 | 0.037 | 80.66/5.68 | | | 810 | 23 | 16 |
| 1897 | Cleaved Peroxisomal | −3.61 | 0.0016 | 38.60/8.78 | 33.59/8.6 | IPI00019912 | 298 | 9.92 | 5 |
| | multifunctional enzyme | | | | | | | | |
| | type 2 (3R)-hydroxyacyl-Co | | | | | | | | |
| | A dehydrogenase) | | | | | | | | |
| 2892 | Phosphatidylethanolamine- | 4.18 | 0.004 | 23.53/8.59 | 21.05/7.01 | IPI00219446 | 243 | 26 | 4 |
| | binding protein 1 | | | | | | | | |
| 1975 | Alpha-2-glycoprotein 1, | −2.24 | 0.0062 | 38.70/5.41 | 34.26/5.71 | IPI00166729 | 112 | 26 | 9 |
| | zinc precursor | | | | | | | | |
| 805 | Polymeric immunoglobulin | 2.08 | 0.0026 | 86.63/5.24 | 83.260/5.59 | IPI00004573 | 238 | 7.3 | 6 |
| | receptor | | | | | | | | |
| 2442 | cDNA FLJ75207 | −2.22 | 0.00047 | 31.15/7.35 | 29.12/8.27 | IPI00004798 | 297 | 12 | 3 |
| 2436 | | −2.45 | 0.0025 | 31.12/7.0 | | | 326 | 12 | 4 |
| 3013 | Prolactin-inducible protein | −3.15 | 0.029 | 16.23/5.01 | 16.56/8.26 | IPI00022974 | 278 | 26 | 3 |
| 3027 | | −2.76 | 0.0053 | 8.85/4.63 | | | 172 | 4 | 2 |
| 1146 | Protein-glutamine gamma- | −1.95 | 0.019 | 67.23/7.24 | 76.62/5.61 | IPI00300376 | 727 | 23 | 10 |
| | glutamyltransferase E | | | | | | | | |
| 2892 | Neutrophil gelatinase-associated | 4.18 | 0.004 | 23.53/8.59 | 22.59/9.02 | IPI00299547 | 503 | 26 | 8 |
| | lipocalin | | | | | | | | |
| 2181 | N-acetylglucosamine kinase | −1.47 | 0.026 | 34.70/5.70 | 37.36/5.82 | IPI00296526 | 1014 | 30 | 14 |
| 1975 | cDNA FLJ53019, highly similar | −2.24 | 0.0062 | 38.70/5.41 | 45.28/5.56 | IPI00006560 | 830 | 26 | 9 |
| | to Serpin B13 | | | | | | | | |
| 1146 | cDNA FLJ54957, highly similar | −1.95 | 0.019 | 67.23/7.24 | 68.74/7.58 | IPI00643920 | 258 | 12 | 5 |
| | to transketolase | | | | | | | | |
| 1203 | | −2.16 | 0.0087 | 65.50/7.62 | | | 222 | 8.7 | 3 |
| 1694 | Placental protein 11 | −1.49 | 0.017 | 49.35/5.23 | 42.10/5.26 | IPI00006995 | 368 | 14 | 4 |
| 1173 | Isoform 1 of heat shock | −2.37 | 0.018 | 65.92/6.3 | 70.88/5.37 | IPI00003865 | 202 | 8.2 | 4 |
| | cognate 71 kDa protein | | | | | | | | |
| 2425 | s-formylglutathione hydrolase | −2.49 | 0.00027 | 31.88/6.6 | 31.45/6.54 | IPI00411706 | 86 | 7.45 | 2 |
| | (esterase) | | | | | | | | |
| 2932 | Small proline-rich protein 3 | 2.37 | 0.014 | 20.47/8.34 | 18.15/8.86 | IPI00082931 | 89 | 28 | 4 |

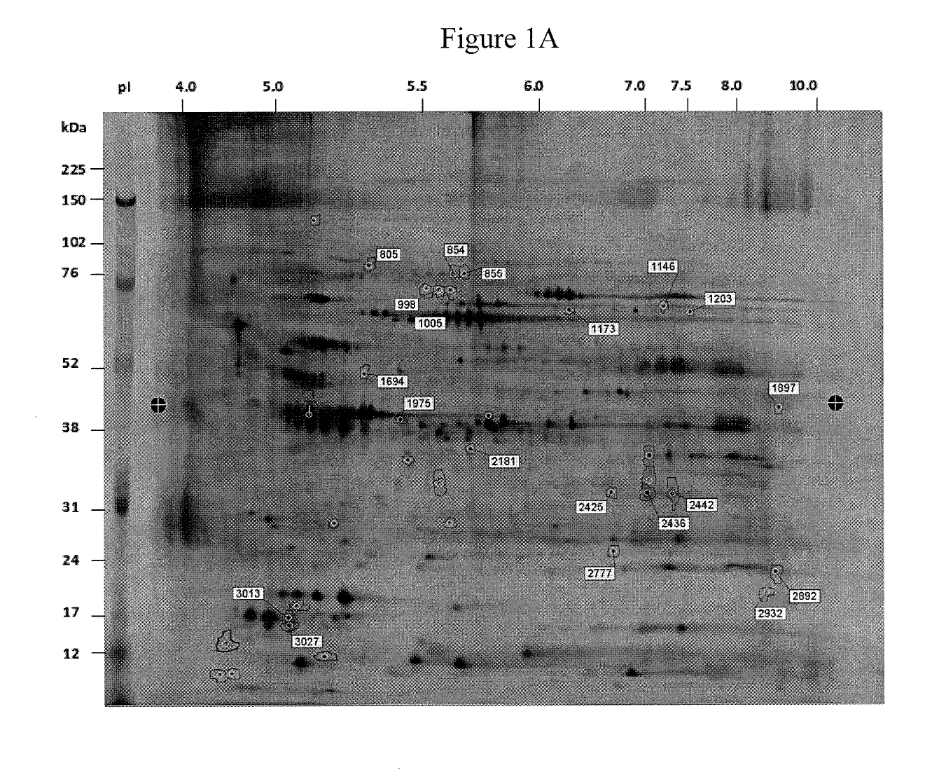

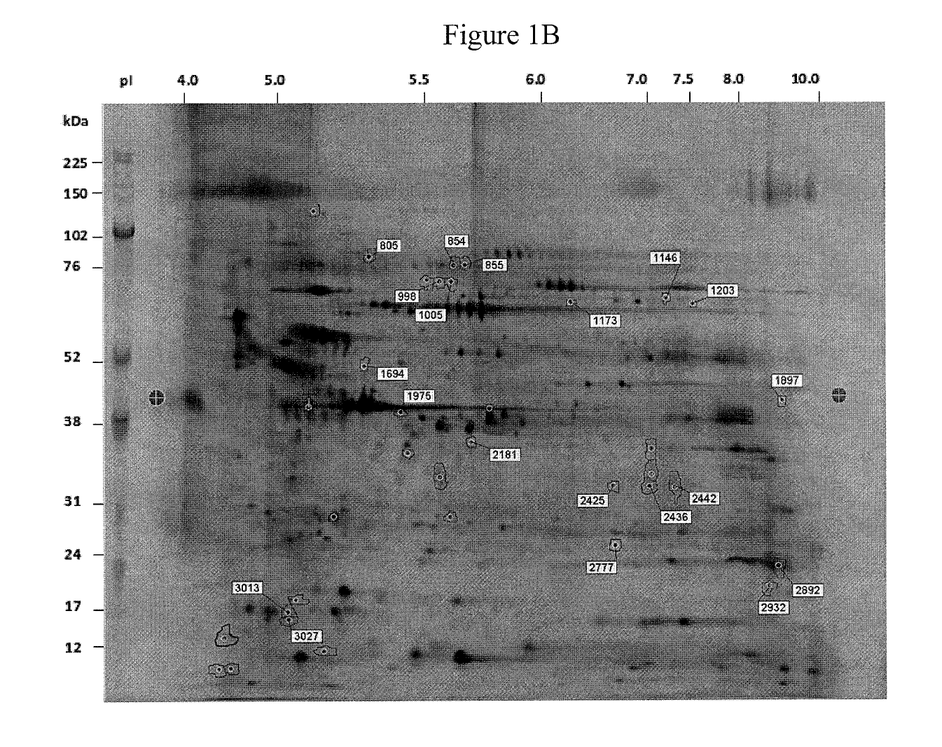

Table I depicts the identification of proteins and their change in NEC infants when compared to normal infants. The spots in Table 1 are listed corresponding to the numbers on the gel image in FIG. 1, which were identified by LC-MS/MS and database searching. The score, percentage of coverage, unique peptides, and theoretical MW and pI were taken from the Mascot report. The experimental MW and pI, and quantitative information were obtained from DeCyder gel image analysis of three replicate gels for each sample.

### Protein Spot Excision

The ordinance information obtained from DeCyder software for each interesting protein spot was transferred to an automated ProPic spot picker (Genomic Solutions) using the pick list. The spots then were excised by the picker and transferred to a collecting plate and were submitted for protein identification.

### Protein Identification

Protein identification was performed using LC-MS/MS. The enzymatically digested samples were injected onto a capillary trap (LC Packings PepMap) and desalted for 5 min with a flow rate of 3 of 0.1% v/v acetic acid. The samples were loaded onto an LC Packing® C18 Pep Map nanoflow HPLC column. The elution gradient of the HPLC column started at 3% solvent A, 97% solvent B and finished at 60% solvent A, 40% solvent B for 30 minutes for protein identification. Solvent A consisted of 0.1% v/v acetic acid, 3% v/v ACN, and 96.9% v/v H2O. Solvent B consisted of 0.1% v/v acetic acid, 96.9% v/v ACN, and 3% v/v H2O.

LC-MS/MS analysis was carried out on a LTQ Orbitrap XL mass spectrometer (Thermo Scientific, Bremen, Germany). The instrument, under Xcalibur 2.07 with LTQ Orbitrap Tune Plus 2.55 software, was operated in the data dependent mode to automatically switch between MS and MS/MS acquisition. Survey scan MS spectra (from m/z 300-2000) were acquired in the orbitrap with resolution R=60,000 at m/z 400. The five most intense ions were sequentially isolated and fragmented in the linear ion trap by collisionally induced dissociation (CID) at a target value of 5,000 or maximum ion time of 150 ms. Dynamic exclusion was set to 60 seconds. Typical mass spectrometric conditions include a spray voltage of 2.2 kV, no sheath and auxiliary gas flow, a heated capillary temperature of 200 degrees C., a capillary voltage of 44V, a tube lens voltage of 165V, an ion isolation width of 1.0 m/z, a normalized CID collision energy of 35% for MS2 in LTQ. The ion selection threshold was 500 counts for MS2. An activation q=0.25 and activation time of 30 ms were set.

### Protein Search Algorithm

All MS/MS spectra were analyzed using Mascot (Matrix Science, London, UK; version 2.2.2). Mascot was set up to search the International Protein Index (IPI) human database assuming the digestion enzyme trypsin. Mascot was searched with a fragment ion mass tolerance of 0.50 Da and a parent ion tolerance of 15 ppm. Iodoacetamide derivative of Cys, deamidation of Asn and Gln, oxidation of Met, are specified in Mascot as variable modifications. Scaffold (version Scaffold-02-03-01, Proteome Software Inc., Portland, Oreg.) was used to validate MS/MS based peptide and protein identifications. Peptide identifications were accepted if they could be established at greater than 95.0% probability as specified by the Peptide Prophet algorithm (Keller, A., Nesvizhskii, A. I., Kolker, E. & Aebersold, R. Empirical statistical model to estimate the accuracy of peptide identifications made by MS/MS and database search. Anal Chem. 2002; 74, 5383-5392). Protein identifications were accepted if they could be established at greater than 99.0% probability and contained at least 2 identified unique peptides. Protein probabilities were assigned by the Protein Prophet algorithm (Nesvizhskii, A. I., Keller, A., Kolker, E. & Aebersold, R. A statistical model for identifying proteins by tandem mass spectrometry. Anal Chem. 2003; 75, 4646-4658).

### Western Blot Analysis

The same protein extracts that were used for 2D-DIGE were also analyzed for Western blotting. Equal amounts of protein samples (15 μg) were loaded to each well and separated on 4-20% SDS-PAGE gels (Bio-Rad Laboratories, Hercules, Calif.) and transferred to Hybond-LFP PVDF membranes (GE Healthcare, Piscataway, N.J.). To check the total protein loading and transferring, blots were stained with SYPRO RUBY Protein Blot Stain (Bio-Rad Laboratories) according to the manufacturer's instructions. A Typhoon Trio+ Variable Mode Imager (GE Healthcare, Piscataway, N.J.) was used to capture the images using a 610 BP 300 Deep Purple filter and 532 nm laser.

Blots were then blocked in 2% ECL Advance blocking agent (GE Healthcare, Piscataway, N.J.) in washing buffer (TBS+0.1% TWEEN) at 4° C. overnight. Three separate blots were probed using different primary antibodies (all 1:1000, Abcam, Cambridge, Mass.), rabbit anti-IL-1RA antagonist, rabbit anti-peroxidorexin 1 or mouse anti-alpha 1 antitrypsin, respectively (1.5 h, RT). Secondary antibodies, ECL Plex goat anti-rabbit Cy5 or ECL Plex goat anti-mouse Cy5 (1:3000, GE Healthcare, Piscataway, N.J.) were incubated with the blots (1 h, RT). Images of blots were captured by a Typhoon Trio+ Variable Mode Imager (GE Healthcare, Piscataway, N.J.) using a 670 BP30 filter and 633 nm laser. Images were analyzed using ImageQuant software (GE Healthcare, Piscataway, N.J.). Relative protein concentrations were quantitated and normalized by total protein loading with SYPRO RUBY staining (Aldridge, G. M., Podrebarac, D. M., Greenough, W. T. & Weiler, I. J. The use of total protein stains as loading controls: an alternative to high-abundance single-protein controls in semi-quantitative immunoblotting. J Neurosci Methods 2008; 172, 250-254).

### Results

Available buccal samples from NEC study cohort (Mai V, Young C M, Ukhanova M, Wang X, Sun Y, Casella G. et al. Fecal microbiota in premature infants prior to necrotizing enterocolitis. PLoS One 2011; 6, e20647) were identified. A total of 3 consecutive buccal samples from 3 NEC cases and 3 closely matchednon-NEC controls were chosen for the analysis. Initial analysis suggested that sufficient amounts of protein would require pooling of weekly samples, thus patients who had 3 samples available prior to the onset of NEC were chosen. 2-D differential gel analysis of NEC baby buccal swab protein levels yielded a total of 37 altered differential protein spots compared to normal control babies; however, only 20 spots were successfully mapped as shown as a representative gel in FIG. 1. These spots were identified as listed in Table 1. A representative example of the mass spectra for one of the proteins of interest (interleukin-1 receptor antagonist, IL-RA) is shown in the supplementary material as FIG. 5.

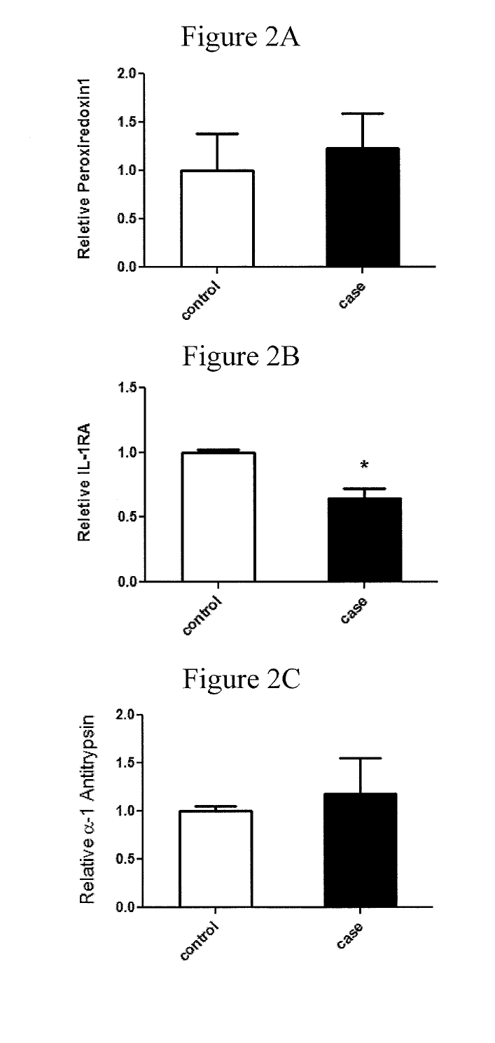

To confirm the findings from the 2-D gel analysis, 3 target proteins were chosen for a Western based analysis. Of the altered proteins identified, peroxiredoxin 1, IL-1RA, and alpha-anti-trypsin were selected as potential candidates for putative predictive markers of NEC. Selection of these proteins was based on their differences seen on the gels and their quality as seen in the mass spectrometry analysis, their physiologic roles as potentially related to NEC and the commercial availability of antibodies. IL-1RA provided the best results, being significantly lower in the cases versus the controls, a p=0.01 (FIG. 2).

A comprehensive bioinformatics analysis was then performed to explore if the 2D based findings pinpoint specific pathways or classes of proteins altered in NEC. The PANTHER analysis (Protein ANalysis THrough Evolutionary Relationships) utilizing the rat protein ontology database, was used to classify proteins into distinct categories of molecular functions and biological processes.

Thirty-eight assignments were obtained for molecular function and were sorted into 6 categories (FIG. 3A). Similarly, 80 assignments were obtained for biological processes and sorted into 12 biological process classifications (FIG. 3B). This is due to the fact that some proteins may be assigned more than 1 molecular function and biological process shown in FIGS. 3A/3B. The percentages listed are calculated as the number of proteins associated with a particular functional block normalized to the total number of proteins.

Cellular localization of the differential proteins is further defined utilizing Pathway Studio version 8.0 (2011, Ariadne Genomics, Rockville, Md.). Although 37 (increased and decreased) unique proteins were detected, a total of 124 hits was assigned in 20 different cellular localizations (FIG. 3C). Note that a protein may be represented in more than one category. Using this approach, it was found that altered proteins belonging to different structural and functional families are regulating different biological processes in the development of NEC (FIG. 3B). The localization of these altered proteins within the cell is shown in FIG. 3C.

Pathway Studio 8.0 (2011, Ariadne Genomics, Rockville, Md.) was also used to search for possible altered cell processes, and related pathways for associations with alterations in the identified proteins from the NEC samples. The network was generated using the “direct interaction” algorithm with the filters of “cellular process; entity and relation type as regulation” to map altered pathways regulated by the identified (increased and decreased) proteins. Several processes believed to be central to the pathogenesis of NEC were identified using this search. Major pathways relevant to NEC development include inflammation, oxidative stress, cell migration, and apoptosis. A global depiction of this interaction network is shown in FIG. 4.

Individual enriched protein-cellular biological process interactions are shown in FIG. 5. The red color shows up-regulated proteins prior to NEC onset. The green color shows proteins that are down-regulated. The shape of a given protein is indicative of its functional class as shown in the legend. Also included in the legend is the definition of the lines connecting 2 proteins. Based on this interaction map, a number of endogenously altered proteins converge on the biological process of inflammatory response, oxidative stress and chemotaxis. The global interaction map depicts the overall pathway changes; a detailed analysis of each specific pathway and its components is shown in supplementary FIG. 5. Of the identified proteins, 13 were shown to regulate the inflammatory related processes (complement activation, neutrophil accumulation and inflammation activation); among these proteins are the three proteins subsequently validated by Western blot (peroxiredoxin 1, IL-1RA, and alpha 1 anti-trypsin) and by MS/MS (see FIG. 6A identifying IL-1RA and FIG. 6B identifying peroxiredoxin). Another relevant pathway includes oxidative stress response, where this process harbors a total of 13 proteins (7 increased and 6 decreased) contributing to the dynamics of oxidative stress either by inhibiting or activating its occurrence as shown in FIG. 5.

### DISCUSSION

This study resulted in the identification of proteins that differed prior to the onset of NEC and, thus, can serve as predictive biomarkers. Investigation of the proteins that differred between NEC and control patients after electrophoresis and MS included evaluating the protein class, molecular and biological functions, and cellular localization of identified proteins. A map identifying the networks and pathways of proteins of interest was subsequently created and evaluated. Finally, to evaluate the validity of the study's approach and confirm the proteomic results, western blots were performed on 3 proteins believed to be the best targets as putative biomarkers of NEC, including peroxiredoxin 1, IL-1RA, and alpha 1 anti-trypsin. Peroxiredoxin is an antioxidant that reduces hydroxyperoxides and peroxynitrites (Van Haver E R, Sangild P T, Oste M, Siggers J L, Weyns A L, Van Ginneken C J. Diet-dependent mucosal colonization and interleukin-1 beta responses in preterm pigs susceptible to necrotizing enterocolitis. Pediatr Gastroenterol Nutr. 2009; 49:90-98). Alpha-1-antitrypsin is a protease inhibitor that protects tissues from enzymes of the inflammatory cells such as neutrophil elastase (Chrystal, R G. The alpha-1-antitrypsin gene and its deficiency states. Trends Genetics 1990; 5:411-417). IL-1RA binds the cell surface IL-1 receptor, hence preventing IL-1 from sending a proinflammatory signal to that cell. IL-1B is especially germane to the pathophysiology of NEC because it causes an increase in intestinal epithelial tight junction permeability (Al-Sadi R M, Ma T Y. IL-1beta causes an increase in intestinal epithelial tight junction permeability. J Immunol. 2007; 178:4641-4649), which is thought to be an antecedent to NEC. Furthermore, IL-1B has been found associated with NEC lesions in a piglet model fed with formula rather than mothers' milk (Rhee, S G, Choe H Z, Kim K. Peroxiredoxins: a historical overview and speculative preview of novel mechanisms and emerging concepts in cell signaling Free Radic Biol Med, 2005; 38:1543-1552). IL-1RA was significantly decreased prior to the onset of NEC in cases when compared to controls, suggesting that low levels of this particular protein may play an active role in NEC and may serve as a candidate biomarker for additional validation for the prediction of infants at risk for NEC.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

I claim:

1. A method for diagnosing necrotizing enterocolitis (NEC) in an infant, wherein said method comprises: (a) obtaining a buccal sample from an infant patient; (b) analyzing the sample to detect at least one protein biomarker of NEC; and, based on the detection of the at least one protein biomarker, (c) diagnosing NEC in the patient.

2. The method according to claim 1, further comprising the step of treating the NEC.

3. The method according to claim 1, wherein the infant patient is a neonate.

4. The method according to claim 1, wherein the infant patient is a preterm infant.

5. The method according to claim 1, wherein detection of the at least one protein biomarker includes determining a level of the at least one protein biomarker in the sample.

6. The method according to claim 1, wherein detection of the at least one protein marker is determined by an immunoassay.

7. The method according to claim 6, wherein the immunoassay is an ELISA assay.

8. The method according to claim 1, wherein the at least one protein biomarker is selected from the group consisting of: peroxiredoxin, IL-IRA, and alpha 1 anti-trypsin.

9. The method according to claim 1, wherein the at least one protein biomarker is IL-1RA.

10. The method according to claim 1, further comprising the step of conducting at least one further diagnostic test for NEC to confirm diagnosis of NEC in the patient.

11. The method according to claim 10, wherein the diagnostic test for NEC is selected from the group consisting of: analysis of intestinal microbiota; analysis of urinary intestinal fatty acid binding protein (I-FABP), claudin-3, and calprotectin; white blood cell counts; platelet counts; abdominal x-rays; and blood cultures.

12. A method for diagnosing and treating NEC in an infant, wherein said method comprises: (a) obtaining a buccal sample from an infant patient; (b) analyzing the sample to detect at least one protein biomarker of NEC selected from the group consisting of peroxiredoxin, IL-1 RA, and alpha 1 anti-trypsin; and, based on the detection of at least one of these protein biomarkers, (c) diagnosing NEC in the patient, and (d) treating the patient diagnosed with NEC.

13. The method according to claim 12, wherein the infant patient is a neonate.

14. The method according to claim 12, wherein the infant patient is a preterm infant.

15. The method according to claim 12, wherein the at least one protein biomarker is IL-1RA.

16. A method for determining an infant's likelihood of developing NEC, comprising: (a) obtaining a buccal sample from an infant patient; (b) analyzing the sample to detect at least one protein biomarker of NEC selected from the group consisting of peroxiredoxin, IL-1RA, and alpha 1 anti-trypsin; and, based on the detection of the at least one protein biomarker, (c) determining the infant's likelihood of developing NEC.

17. The method according to claim 16, wherein the infant patient is a neonate.

18. The method according to claim 16, wherein the infant patient is a preterm infant.

19. The method according to claim 16, wherein the at least one protein biomarker is IL-1RA.

20. The method according to claim 16, further comprising the step of treating the infant.
